(19) United States
(12) Patent Application Publication
(10) Pub. No.: US 2023/0218802 A1
(43) Pub. Date: Jul. 13, 2023

(54) PRODUCTION OF XENOGRAFT FROM ANIMAL BONES

(71) Applicant: YILDIZ TEKNIK UNIVERSITESI, Istanbul (TR)

(72) Inventors: Cem Bulent USTUNDAG, Istanbul (TR); Davut YERLIKAYA, Istanbul (TR); Burcu Tugba SIMSEK, Istanbul (TR); Ahmet Goktug TUFEK, Istanbul (TR); Nurettin DIKER, Istanbul (TR)

(73) Assignee: YILDIZ TEKNIK UNIVERSITESI, Istanbul (TR)

(21) Appl. No.: 18/009,799

(22) PCT Filed: Dec. 7, 2020
(86) PCT No.: PCT/IB2020/001062

§ 371 Date: Dec. 12, 2022

(30) Foreign Application Priority Data
Jun. 11, 2020 (TR) 2020/09080

(51) Int. Cl. A61L 27/36

(52) U.S. Cl. CPC A61L 27/3608; A61L 27/3687; A61L 27/3691; A61L 27/365; A61L 2430/02; A61L 2430/40

(57) ABSTRACT

A method for producing xenogeneic bone graft, which is a material for use in bone tissue therapy, is provided. The method includes the steps of isolating the cancellous bones from cartilage and cortical bone by fragmenting them into pieces, washing the bone fragments with purified water to partially remove the organic phases and boiling them with purified water, contacting the bone fragments with a solvent for further removal/isolation of the organic phase, washing the bone fragments to remove the solvents used and other possible residues, subjecting them to a hydrothermal and/or solvothermal treatment with a solvent at a pressure of more than 1 atm and a temperature in the range of 100° C. to 300° C., after which washing them with purified water, and drying the cancellous bone fragments, for example, at a temperature in the range of 50° C. to 100° C. to dehydrate them.

### CROSS REFERENCE TO THE RELATED APPLICATIONS

This application is the national phase entry of International Application No. PCT/IB2020/001062 filed on Dec. 7, 2020, which is based upon and claims priority to Turkish Patent Application No. 2020/09080 filed on Jun. 11, 2020, the entire contents of which are incorporated herein by reference.

### TECHNICAL FIELD

The present invention relates to a method for producing xenograft from animal bones. In particular, the present invention relates to the retention of size and porosity in such a method.

### BACKGROUND

For various reasons, people either lose their bone tissues, or their bone tissues are damaged. Some congenital diseases may also cause deficiency or deformation in bone tissues. Especially in dentistry, after tooth extraction, resorption occurs in the alveolar bone holding the teeth, which is followed by gingival recession in that area. The loss of alveolar bone may successively also cause the loss of the healthy teeth in the neighboring area. Furthermore, bone formation in that area should be supported in order for the dental implants to be fitted firmly. The re-formation and treatment of the palatine bone requires the use of an additional material (bone grafts, etc.). In dentistry applications, synthetic or natural materials are used for such purposes. These materials provide an environment where the cells required for the formation of natural bone can hold on, grow and proliferate, thereby allowing regeneration and formation of the bone. With oral and maxillofacial surgery methods, such materials are placed in the defective parts of the jaw bones. The graft materials that are placed are intermediary and pioneering in the formation of new bones that will support the teeth and the dental implants.

Various application areas and forms of bone grafts as a surgical supporting material are disclosed in https://www.geistlich-pharma.com/en/dental/membranes/geistlich-bio-gide-shape/application/ and in Richardson et al. (J Clin Periodontol 1999; 26: 421-428) and Palachur et al. (J Indian Soc Periodontol. 2014 May; 18(3):336-43).

Synthetically produced bioceramic materials containing calcium phosphate are currently used in dental applications for such problems. However, the low ratio of calcium and phosphorus (Ca/P ratio) compared to hydroxyapatite in the natural bone structure, and long biodegradation times due to high crystallinity reduce the attractiveness of these calcium phosphate-containing materials. At this point, the use of xenogeneic bone grafts may also be preferred due to their similarity to the natural bone tissue. Various techniques are known for obtaining bone grafts from cancellous bones of animals. However, these methods are inadequate due to the level of purity they provide, the inability to preserve the natural structure of the bone, and their biodegradation times.

U.S. Pat. No. 5,167,961 discloses a process for preparing high purity bone mineral. US 2003/0074065 A1 relates to bone xenografts. WO 2013/008959 A1 describes a xenograft-derived bone grafting substitute, and a method for manufacturing same.

In the methods that are widely used in the prior art, animal cancellous bone is isolated from organic components by chemical processes and a heat treatment is applied at a temperature in the range of 250° C. to 600° C. A cancellous bone graft is obtained, which is well decellularized and isolated from organic components. The resulting cancellous bone preserved its natural porous structure and inorganic part, but the internal surface area of the bone was reduced.

A cancellous bone skeleton is obtained by boiling the cancellous bone with various chemicals and purified water, followed by firing at high temperatures such as 1000° C. After the bones were treated with chemical materials such as chloroform and sodium hypochlorite, they were subjected to heat treatment at high temperatures, as a result of which a chloroapatite form was also observed in the structure as well as hydroxyapatite (Jung et al., J Biomed Mater Res Part B 2013:101B:855-869). Due to the heat treatment (firing) performed at very high temperatures, the structure of hydroxyapatite in the natural bone structure is disrupted and crystallinity increases. Therefore, a bone graft with the desired level of biodegradation cannot be obtained.

In addition to the boiling process to partially defat them, firing of the bones that have been previously powdered at temperatures higher than 700° C. is performed, without further chemical processing. Bones are subjected to a heat treatment at high temperatures in order to isolate the organic phase, which cannot be removed to a greater extent by chemical processes. Therefore, carbonate groups are formed in the structure, and much higher temperatures are required to remove them. In addition, with the heat treatment applied, the grain size of the graft grows (crystallinity increases), and accordingly the biodegradation time increases.

Bones are subjected to chemical treatments before and after they are pulverized. Following said chemical processes, the powdered bones are subjected to a heat treatment at high temperatures. The bone, which is pulverized during the chemical processes, cannot protect its porous structure. It causes additional cost to obtain a graft again having a porous structure, from the powdered material. Due to the heat treatment at high temperatures, the natural form of hydroxyapatite starts to transform into TCP (tri calcium phosphate), which is undesirable in bone graft.

In xenogeneic bone grafts produced so far, the natural structure of the bone can only be preserved to a limited extent, and the biodegradation time is inevitably prolonged with the increase in crystallinity resulting from the heat treatments. The long-term chemical process used in the previous methods causes high costs and loss of the natural structure of the bone. The loss of the natural structure of the bone causes the bone graft to be less durable and unable to create a suitable environment for cells. Furthermore, the heat treatments at high temperatures that are carried out during decellularization cause a high crystallinity of the bone grafts obtained and a very slow biodegradation thereof. For all these reasons, there is still a need for further improvement of the methods for producing porous xenografts from animal bones.

### SUMMARY

The main object of the invention is to provide solutions to the prior art problems. Another object of the invention is to provide a method that allows animal bones with cancellous tissue to be transformed into xenogeneic bone grafts without destroying their original porosity. Another object of the invention is to provide a method that allows animal bones with cancellous tissue to be transformed into xenogeneic bone grafts without destroying their original crystalline structure.

The present invention provides a method for producing xenogeneic bone grafts. Said method comprises the step of subjecting the cancellous bones to a hydrothermal and/or solvothermal treatment with a solvent at a pressure of more than 1 atm and a temperature in the range of 100° C. to 300° C. Thus, the Ca/P ratio, the original crystalline structure and the porosity of the cancellous bones used in the preparation of the xenogeneic bone grafts are preserved, by avoiding heavy chemical processes and high temperatures.

### BRIEF DESCRIPTION OF THE DRAWINGS

The drawings, whose brief description is provided below are just given for better understanding of the present invention and as such, are not intended to determine the scope of the claimed subject matter, in the absence of the description.

FIG. 1 is a schematic diagram representing a process step of the present invention and the process steps that can be carried out before and after this process step.

FIG. 2 is a schematic sectional view of an exemplary autoclave reactor suitable for performing the hydrothermal/solvothermal treatment step of the present invention.

Reference numerals: 10: bolt; 20: filter pad; 30: holder; 40: coupling cover; 50: cover; 60: liner; 70: outer shell; 80: solvent(s); and 90: base.

### DETAILED DESCRIPTION OF THE EMBODIMENTS

In the prior art, the problems of the methods used in the production of xenogeneic bone grafts (an increase in the crystallinity of the materials produced due to the heat treatment at high temperatures and consequently the lack of biodegradation in the desired period, the emergence of the TCP (tri calcium phosphate) phase due to the dehydroxylation of the hydroxyapatite in the structure of the bone during the heat treatment process and the degradation of the natural bone structure, a lower Ca/P ratio as compared to the natural bone resulting from the heat treatments, etc.) are overcome by the method of the present invention having a hydrothermal/solvothermal treatment step, in addition to other chemical and mechanical processes. With the hydrothermal/solvothermal treatment, a xenogeneic bone graft may be obtained, which preserves the structure of the natural hydroxyapatite crystal and has rapid biodegradation. The method of the present invention comprises the following process step:
i) subjecting the cancellous bones to a hydrothermal/solvothermal treatment with a solvent at a pressure of more than 1 atm and a temperature in the range of 100° C. to 300° C.

Said process step may be regarded as hydrothermal when water is used as a solvent, and as solvothermal when another solvent is used. If one or more solvents are present in the process, it can be referred to as a hydrothermal and solvothermal treatment step. Suitable solvents for the hydrothermal/solvothermal treatment step may include various solvents such as water (preferably purified water), sodium hypochlorite, chloroform, acetone, and other organic, inorganic, and ionic solutions (salt solutions, etc.), or a mixture thereof. In other words, suitable solvents for the hydrothermal/solvothermal treatment step may be selected from the following: organic solvents, inorganic solvents, ionic solutions, and mixtures thereof. The hydrothermal/solvothermal treatment is preferably continued for a period of 6 hours to 24 hours. In the hydrothermal/solvothermal treatment step, the grain size does not change, because high temperatures that have to be reached in the prior art are avoided, and the natural crystalline structure, dimensions and porosity of the cancellous bone fragments can be preserved, without degradation.

By adapting the above-described step (i) instead of the heavy chemical processes and/or the process steps requiring high temperatures in the prior art methods, a method allowing production of xenogeneic grafts from animal bones with a cancellous structure may be thoroughly designed by a skilled person in the art.

Preferably, the present invention may comprise the following steps in sequence, prior to step (i):
a) Obtaining cancellous bone fragments by crushing an animal bone with cancellous tissue (e.g., head of femoral bone-femur head). Accordingly, the cancellous part of the bone can be isolated from the cartilage and cortical bone, so the cancellous bone fragments, preferably in the order of 0.5-1.5 centimeters, can be obtained.
b) Partial removal of the organic phases in the cancellous bone fragments (e.g., partial removal/isolation of fats, blood, and other unwanted organic phases). Accordingly, for example, the cancellous bone fragments may be washed with purified water 10-15 times for 1-2 hours and boiled 3-5 times with purified water for 30-60 minutes.
c) Further removal/isolation of the organic phase of the cancellous bones. Accordingly, the cancellous bones may be contacted with a solvent capable of dissolving organic components (e.g., proteins, fats, DNA, cells, etc.); this step may be referred to as “chemical treatments step” and “decellularization”. In this step, they are contacted with the solvents that will dissolve the organic phase (fats, proteins, etc.) and remove it from the cancellous bones, such as sodium hypochlorite, chloroform, acetone, or mixtures thereof, additionally or also other organic, inorganic and ionic solutions (salt solutions, etc.) or mixtures thereof, for a period of e.g. 7-15 days, and it is preferably stirred in a continuous manner.
d) Washing of the cancellous bone fragments. With the washing step, the cancellous bone fragments are removed from the solvent(s) mentioned in step (c) and other possible residues. Said washing process may be repeated 10-15 times for 1-2 hours, for example with purified water.

In addition to the above steps, the present invention may comprise the following steps in sequence, after step (i):
e) Washing of the cancellous bone fragments. Thus, any residues that may still present on the cancellous bone fragments are removed. Said washing process may be carried out, for example, with water, e.g., purified water. The washing process may be repeated for a period of e.g., 1 to 7 days, and e.g., 3 or more times.
f) Dehydration of the cancellous bone fragments by drying. Said drying process can be carried out, for example, at a temperature in the range of 50° C. to 100° C., and for a period of time in the range of for example 1 to 2 days.

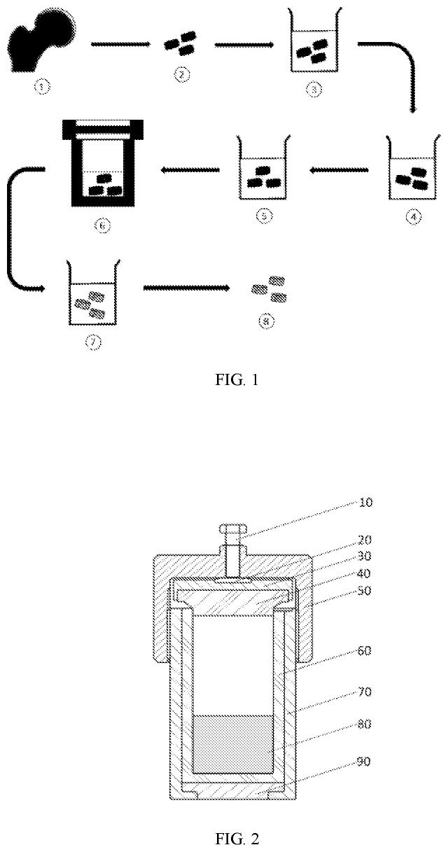

FIG. 1 is a diagram depicting the possible steps of the method according to the invention. In step 1, the original version of the head of an exemplary animal femoral bone is illustrated. Step 2 represents the isolation of the cancellous bone from the tendon, cartilage, and cortical bone by fragmenting the head of the femoral bone into small pieces. Step 3 represents the washing of the cancellous bone fragments several times in purified water and boiling them with purified water, in order to partially defat same. In step 4, the decellularization of the cancellous bone fragments by means of a chemical process and the removal of the organic residues such as fat, etc. is represented.

Step 5 represents further removal of the chemical residue and dissolved organic materials by washing the cancellous bone fragments with purified water. Step 6 is directed to the hydrothermal/solvothermal treatment step of the method according to the present invention, wherein it is represented that the bone fragments are treated at a temperature in the range of 100° C. to 300° C. and under a pressure above 1 atm, preferably for a period of 6 hours to 24 hours. In step 7, the washing process (e.g., with purified water) is represented to remove any organic excess that is likely to form residue on the cancellous bone fragments after the hydrothermal/solvothermal treatment step. In step 8, it is represented to obtain xenogeneic bone grafts with preserved natural crystalline structure and porosity by drying the obtained purified cancellous bone fragments.

FIG. 2 is a schematic sectional view of an exemplary autoclave reactor suitable for performing the hydrothermal/solvothermal treatment step of the present invention. A known commercially available device can be used as a reactor in the hydrothermal/solvothermal step. For example, as of the date of application, it is available from TOPTION under the name of “Teflon lined hydrothermal autoclave reactor” (see FIG. 2, source: https://www.toptiontech.com/info/how-to-use-hydrothermal-autoclave-reactor-9587371.html). Said reactor may comprise a liner (60) surrounded by an outer shell (70) and a base (90), and acting as a reservoir through which one or more solvents (80) are brought into contact with the cancellous bone fragments. The liner (60) may be made of PTFE, for example. There may be a coupling cover (40) to cover an inlet opening located on the liner (60), and a holder (30) that grips it. The coupling cover (40) may preferably be made of the same material as the liner (60) so that it does not corrode the liner. In order to tightly couple the holder (30) with the outer shell (70) against the high pressures that can be reached in the hydrothermal/solvothermal treatment step, the reactor may include a cover (50) that surrounds the holder (30) and the outer shell (70) from the outside, and that is attached to the holder (30), for example by means of a bolt (10), and to the outer shell by means of a threaded perimeter. A filter pad (20) may be located between the bolt (10) and the holder (30). In order to be resistant to high pressures, the holder (30), the cover (50), the outer shell (70) and the base (90) may be made of steel.

The hydrothermal/solvothermal method may also be performed at temperatures above 300° C. if the reactor is not PTFE-lined, but higher temperatures are not required as the temperatures in the range of 100° C. to 300° C. yield excellent results. Therefore, there is no obstacle to the use of the reactors that can be easily cleaned by virtue of being PTFE-lined. Thus, the method makes it easy to clean the reactor after the hydrothermal/solvothermal treatment step.

In the method, chemical processes applied on the cancellous bone fragments to remove proteins and other organic components are both less in number and less damaging. In addition, the reactor temperatures in the hydrothermal/solvothermal treatment step (step i) forming the basis of the method are lower than those required by the prior art methods. Thus, white cancellous bone fragments (xenogeneic bone grafts) can be obtained, which preserve its natural inorganic structure and are free of organic components. The resulting cancellous bone fragments have a lower crystallinity (relatively small grain size) than those provided in the prior art.

The block and particle forms of the bone graft material to be produced using the cancellous bone fragments provided by the method of the present invention are suitable for use in dentistry and various orthopedic applications.

The xenogeneic bone graft material produced with the hydrothermal/solvothermal treatment step of the inventive method is industrially producible by suppling appropriate equipment and materials and thus is industrially applicable. Thanks to the inventive improvement, the deficiencies of the prior art have been eliminated and the aforementioned problems have been solved.

What is claimed is:

1. A method for producing xenogeneic bone grafts, comprising subjecting cancellous bones to a hydrothermal and/or solvothermal treatment with a first solvent at a pressure of more than 1 atm and a temperature in a range of 100° C. to 300° C.

2. The method according to claim 1, wherein the first solvent is selected from the group consisting of organic solvents, inorganic solvents, ionic solutions, and mixtures of the organic solvents, the inorganic solvents, and the ionic solutions.

3. The method according to claim 2, wherein the first solvent is selected from purified water, sodium hypochlorite, chloroform, acetone, or mixtures of the purified water, the sodium hypochlorite, the chloroform, and the acetone.

4. The method according to claim 1, wherein the hydrothermal and/or solvothermal treatment is continued for a period of 6 hours to 24 hours.

5. The method according to claim 1, comprising the following steps in sequence, prior to the hydrothermal and/or solvothermal treatment:
a) obtaining cancellous bone fragments by crushing an animal bone with a cancellous tissue;
b) partially removing of organic phases in the cancellous bone fragments;
c) further removing/isolating of the organic phases of the cancellous bone fragments; and
d) washing the cancellous bone fragments.

6. The method according to claim 5, wherein in the step (b), the cancellous bone fragments are washed with a purified water with a first number of repetitions in a range of 10 to 15 times for a period of 1 hour to 2 hours and boiled with the purified water with a second number of repetitions in a range of 3 to 5 times for a period of 30 minutes to 60 minutes.

7. The method according to claim 5, wherein in the step (c), the cancellous bone fragments are contacted with a second solvent capable of dissolving organic components for a period of 7 days to 15 days.

8. The method according to claim 7, wherein the second solvent being able to dissolve the organic components is selected from organic solvents, inorganic solvents, ionic solutions, and mixtures thereof the organic solvents, the inorganic solvents, and the ionic solutions.

9. The method according to claim 5, wherein the washing in the step (d) is repeated with a purified water with a repetition number in a range of 10 to 15 times for a period of 1 hour to 2 hours.

10. The method according to claim 5, comprising the following steps in sequence after the hydrothermal and/or solvothermal treatment:
e) washing the cancellous bone fragments; and
f) drying the cancellous bone fragments.

11. The method according to claim 10, wherein the washing 9 in the step (e) is performed with a purified water for a period of 1 day to 7 days and repeated 3 or more times.

12. The method according to claim 10, wherein the drying in the step (f) is carried out at a temperature in a range of 50° C. to 100° C. for a period of 1 day to 2 days.

13. The method according to claim 2, wherein the hydrothermal and/or solvothermal treatment is continued for a period of 6 hours to 24 hours.

14. The method according to claim 3, wherein the hydrothermal and/or solvothermal treatment is continued for a period of 6 hours to 24 hours.

15. The method according to claim 6, wherein in the step (c), the cancellous bone fragments are contacted with a second solvent capable of dissolving organic components for a period of 7 days to 15 days.

16. The method according to claim 11, wherein the drying in the step (f) is carried out at a temperature in a range of 50° C. to 100° C. for a period of 1 day to 2 days.

17. The method according to claim 6, comprising the following steps in sequence after the hydrothermal and/or solvothermal treatment:
e) washing the cancellous bone fragments; and
f) drying the cancellous bone fragments.

18. The method according to claim 7, comprising the following steps in sequence after the hydrothermal and/or solvothermal treatment:
e) washing the cancellous bone fragments; and
f) drying the cancellous bone fragments.

19. The method according to claim 8, comprising the following steps in sequence after the hydrothermal and/or solvothermal treatment:
e) washing the cancellous bone fragments; and
f) drying the cancellous bone fragments.)

20. The method according to claim 9, comprising the following steps in sequence after the hydrothermal and/or solvothermal treatment:
e) washing the cancellous bone fragments; and
f) drying the cancellous bone fragments.
